(19) United States
(12) Patent Application Publication
(10) Pub. No.: US 2024/0138448 A1
(43) Pub. Date: May 2, 2024

(54) VACUUM SYSTEM AND METHOD FOR STERILIZATION

(71) Applicant: Leybold GmBH, Cologne (DE)

(72) Inventor: Sabastian Stefan Rosenstraeter, Cologne (DE)

(21) Appl. No.: 18/574,237

(22) PCT Filed: Jul. 1, 2022
(86) PCT No.: PCT/EP2022/068322

§ 371 Date: Dec. 26, 2023

(30) Foreign Application Priority Data
Jul. 2, 2021 (GB) 2109584.9

(51) Int. Cl. A23L 3/3445; A23L 3/00

(52) U.S. Cl. CPC A23L 3/3445; A23L 3/001

(57) ABSTRACT

Vacuum system, in particular for food processing, comprising: a main valve being connectable to a processing chamber, a vacuum pump having an inlet and an outlet, wherein the inlet is connected to the main valve to connect the vacuum pump with the processing chamber, and a sterilization device being connected at a point of connection between the main valve and the inlet, wherein the sterilization device is configured to provide a sterilization gas.

This application is a national stage entry under 35 U.S.C. § 371 of International Application No. PCT/EP2022/068322, filed Jul. 1, 2022 and entitled “VACUUM SYSTEM AND METHOD FOR STERILIZATION,” which claims the benefit of GB Application No. 2109584.9, filed Jul. 2, 2021 and entitled “VACUUM SYSTEM AND METHOD FOR STERILIZATION,” the entire contents of each of which are incorporated herein by reference.

### TECHNICAL FIELD

The present disclosure relates to a vacuum system in particular for food processing as well as a method for sterilization of such a vacuum system.

### BACKGROUND

A lot of processes in the food industry require some form of vacuum. Vacuum systems are used both for the processing of the actual food and for the packaging processes.

### SUMMARY OF THE INVENTION

The food industry has very high hygienic standards to prevent the end customer from receiving food contaminated with harmful bacteria or viruses or molds. Therefore, the production equipment is regularly thoroughly cleaned/sterilized from the inside and outside. If a vacuum system is part of the production equipment, this is usually only cleaned/disinfected from the outside. The interior sterilization usually ends at the main vacuum valve or even before. Any pipes, fittings, valves, and the vacuum pump itself will not be sterilized from the inside. However, tests have shown that typical bacteria and molds harmful to food processes or consumers can survive inside a vacuum system used for food and packaging applications. Although being downstream from the actual food, these phages can possibly contaminate the process itself resulting in product recalls or worse for the producer.

Thus, it is an object of the present disclosure to provide a vacuum system being able to be sterilized as well as a method for sterilizing such vacuum system.

In an aspect of the present disclosure a vacuum system is provided in particular for food processing. The vacuum system comprises a main valve being connectable to a processing chamber of the food process. Further, the vacuum system comprises a vacuum pump having an inlet and an outlet. Therein, the vacuum pump can be built as any common vacuum pump such as screw pumps, claw pumps, rotary vane pumps and the like. The present disclosure is not limited to a specific type of vacuum pump used in the vacuum system. Therein, the inlet of the vacuum pump is connected to the main valve in order to connect the vacuum pump with a processing chamber. Usually, the main valve is connected with the inlet of the vacuum pump via a pipe wherein the main valve having a flange in order to connect the main valve to the processing chamber of the food process. Further, the vacuum system comprises a sterilization device being connected to a point of connection between the main valve and the inlet preferably by a branch connection of the pipe connecting the main valve with the inlet of the vacuum pump. Therein, the sterilization device is configured to provide a sterilization gas. Thus, by operating of the vacuum pump the sterilization gas is conveyed from the sterilization device through the pipe connecting the main valve and the inlet of the vacuum pump, through the vacuum pump towards the outlet of the vacuum pump thereby sterilizing the interior of the vacuum system. Thus, by implementing a sterilization device in the vacuum system and providing a sterilization media in a gaseous form, the interior of the vacuum system can be thoroughly sterilized in order to avoid infections, bacteria mold, and the like.

Preferably, a sterilization valve is disposed between the sterilization device and the point of connection in order to separate the sterilization device from the other parts of the vacuum system and the process chamber during normal operation. Upon sterilization, the sterilization valve is opened to connect the sterilization device with the vacuum pump.

Preferably, the sterilization device is disposed close to the main valve in order to reduce the length of the pipe between the main valve and the connection point connecting the sterilization device with the other parts of the vacuum system. Thus, the sterilization gas is enabled to flow through almost all interior volumes of the vacuum system providing a reliable and almost complete sterilization. Therein, the sterilization device and the main valve might be connected directly next to each other and/or by a common T-branch. Thereby, the length of the pipe between the main valve and the connection point of the sterilization device is reduced to less than 10 cm, more preferably less than 5 cm and most preferably less than 2 cm. However, if the system is evacuated and then the pump stopped, the position of the sterilization device is not limited by the present disclosure. The gas will reach every part of the vacuum system due to diffusion.

Preferably, the sterilization gas is one or more of ozone, fluorine, bromine, iodine and hydrogen peroxide, all in gaseous form.

Preferably, the sterilization gas has a concentration of less than 20%, preferably less than 10% and more preferably less than 2% of the volume of the atmosphere within the vacuum system. Thus, even a low concentration of the sterilization gas is sufficient in order to provide reliable sterilization of the interior of the vacuum system.

Preferably, the sterilization device is an ozone generator configured to generate ozone via a plasma, electrolysis, and electrostatic discharge. Thus, a less complex sterilization device is provided supplying a reliable amount of sterilization gas into the vacuum system. Further, such ozone generators are less expensive and widely available. Sterilization device could also be a dispensing unit that is supplied with the sterilization gas in bottle form or similar.

Preferably, an inlet valve is arranged at the inlet of the vacuum pump wherein preferably the inlet valve is built as gas flow limiter. Thus, by the inlet valve the gas flow through the vacuum system upon the sterilization process can be reduced providing a sufficient length of retention or length of stay of the sterilization gas within the vacuum system in order to provide sterilization. Alternatively or additionally, an outlet valve is arranged at the outlet of the vacuum pump which might be also built as gas flow limiter wherein closing the outlet valve increases the time of retention of the sterilization gas within the vacuum system by reducing the gas flow through the vacuum system. Thus, by arranging the outlet valve at the outlet of the vacuum pump also the interior of the vacuum pump itself can be sufficiently sterilized.

Preferably, a gas filter is arranged at the outlet of the vacuum pump. Thus, the sterilization gas is filtered by the gas filter and cannot leak into the environment.

Preferably, the vacuum pump is built as oil free vacuum pump. Since some of the sterilization gases are highly reactive, implementing the vacuum pump as oil free vacuum pump avoids interaction of the sterilization gas with the lubrication of the vacuum pump of the vacuum system in order to avoid degradation of the lubrication of the vacuum pump. Thus, by implementing the vacuum pump as oil free vacuum pump the lifetime of the vacuum system is increased.

In another aspect of the present disclosure a method for sterilization of a vacuum system is provided. Therein, the vacuum system is preferably built as described above. Therein, the method comprises the steps of closing the main valve in order to separate the food process chamber from the rest of the vacuum system and starting the sterilization device in order to provide a sterilization gas to the vacuum system. Therein, the steps can be performed simultaneously, or subsequently in any order.

Preferably, operation of the vacuum pump is maintained to create a gas flow from the sterilization device towards the vacuum pump and in particular through the vacuum pump to the outlet of the vacuum pump. Thus, the sterilization gas provided by the sterilization device is drawn through the vacuum system and providing sufficient and reliable sterilization of the vacuum system.

Preferably, the pump performance of the vacuum pump is reduced for example by reducing the rotational speed of the vacuum pump in order to reduce the gas flow through the vacuum system. Thus, by reducing the gas flow through the vacuum system time of retention of the sterilization gas within the vacuum system is increased improving the sterilization effect.

Preferably, a sterilization valve is implemented between the sterilization device and the connecting point of the pipe connecting the main valve with the inlet of the vacuum pump separating the sterilization device from the rest of the vacuum system in particular during normal operation. Thus, before sterilization of the vacuum system, after closing the main valve, the sterilization valve is opened in order to enable the sterilization gas to flow from the sterilization device through the vacuum system. Alternatively, opening the sterilization valve and closing the main valve can be performed simultaneously.

Preferably, the gas flow is reduced by at least partially closing the inlet valve being connected to the inlet of the vacuum pump and/or at least partially closing the outlet valve being connected to the outlet of the vacuum pump. Thus, the length of retention of the sterilization gas within the vacuum system is increased improving the sterilization effect.

Preferably, the gas flow is reduced by partially opening the sterilization valve being connected to the connecting point. Thus, the length of retention of the sterilization gas within the vacuum system is increased improving the sterilization effect.

Preferably, the inlet valve is closed completely, thereby avoiding that the sterilization gas is conveyed out of the system. After a predetermined length of retention, the inlet valve is reopened such that new sterilization gas is drawn from the sterilization device into the vacuum system. Preferably, closing and opening the inlet valve are repeated periodically thereby providing sufficient time during the closed inlet valve for the sterilization gas to stay inside the vacuum system and provide a sufficient sterilization effect.

Preferably, the outlet valve is closed completely, thereby avoiding that the sterilization gas is conveyed out of the system. After a predetermined length of retention, the outlet valve is reopened such that new sterilization gas is drawn from the sterilization device into the vacuum system. Preferably, closing and opening the outlet valve are repeated periodically thereby providing sufficient time during the closed outlet valve for the sterilization gas to stay inside the vacuum system and provide a sufficient sterilization effect.

Preferably, the length of retention corresponds to the chemical decay time of ozone. Ozone is a very unstable substance, recombining to molecular oxygen after a certain time. While ozone is harmful to humans and the environment, after recombination to molecular oxygen, the recombined sterilization gas can escape to the environment without harm. Thus, by selecting the length of retention corresponding to the decay time of ozone, it is assured that recombination of the ozone to molecular oxygen mainly happens within the vacuum system.

### BRIEF DESCRIPTION OF DRAWINGS

In the following the present disclosure is described in more detail with reference to the accompanied figures.

FIG. 1 a first example of the present disclosure.

FIG. 2 a second example of the present disclosure.

FIG. 3 a third example of the present disclosure.

FIG. 4 a flow diagram of the method according to the present disclosure.

FIG. 5 a flow diagram of another example of the method according to the present disclosure.

### DETAILED DESCRIPTION

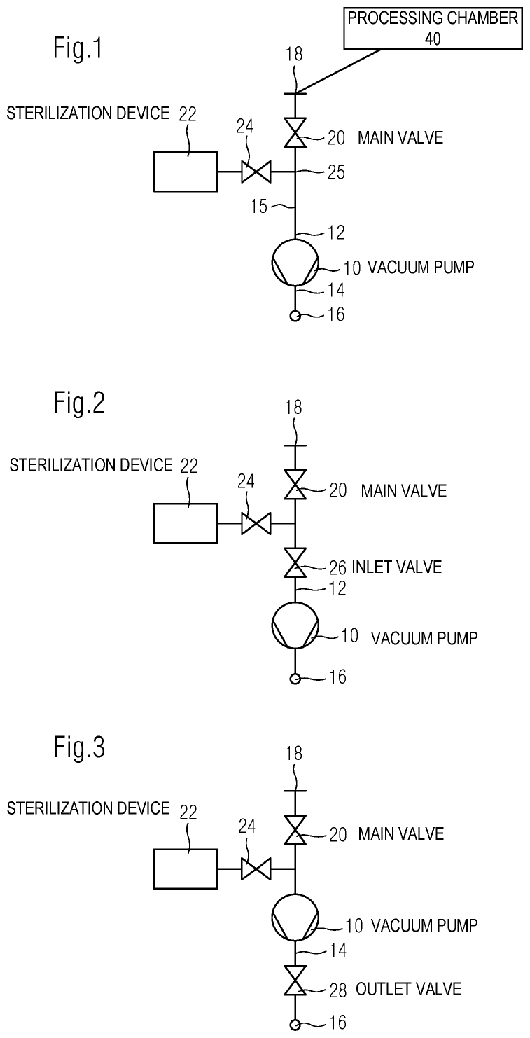

Referring to FIG. 1 showing a vacuum system according to the present disclosure. The vacuum system comprises a vacuum pump 10 having an inlet 12 and an outlet 14. The inlet 12 is connected via a pipe with a main valve 20. Therein, the main valve 20 may comprise a flange 18 or be connected in any other manner to the processing chamber 40 of the vacuum process. Thus, by the vacuum pump 10 a gaseous medium is drawn from the processing chamber 30 connected to the main valve 20 through the pipe 15, through the vacuum pump towards the exhaust 16 of the vacuum system connected to the outlet 14 of the vacuum pump 10. Further, the vacuum system comprises a sterilization device 22 being connected to the pipe 15 at a connection point 25 for example by a branch connection or T-connection. Therein, between the connection point 25 and the sterilization device 22 a sterilization valve 24 is arranged in order to separate the sterilization device 22 from the rest of the vacuum system during normal operation. Further, the connection point 25 is as close as possible to the main valve 20 in order to reduce the length of pipe 15 between the main valve 20 and the connection point 25. Preferably, the distance between the main valve 20 and the connection point 25 is less than 10 cm, preferably less than 5 cm and more preferably less than 2 cm. In this manner, the vacuum system may minimize the distance through pipe 15 in the flow from the sterilization device 22 through the vacuum pump 10 towards the exhaust 16 of the vacuum system.

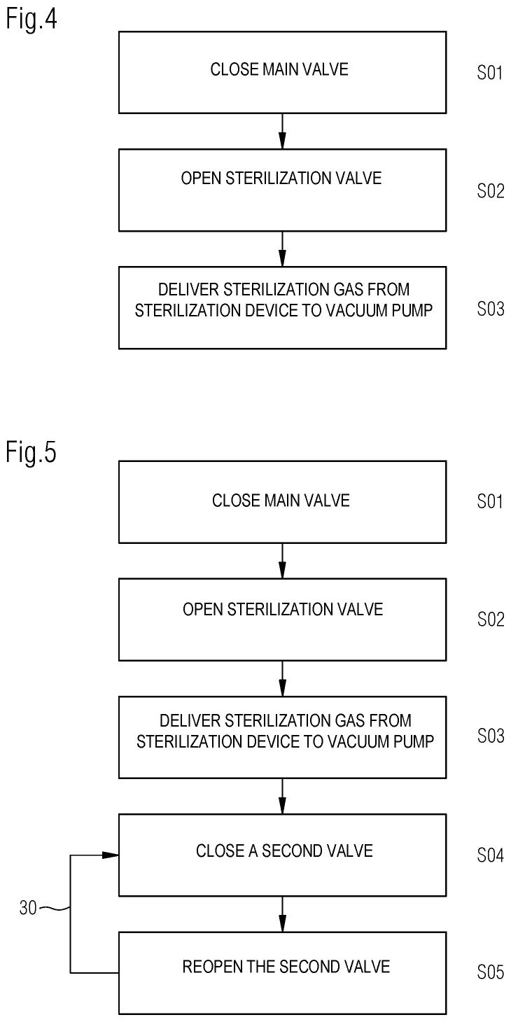

In accordance to the disclosure, by the sterilization device 22 a sterilization gas is provided and drawn by the vacuum pump 10 through the vacuum system in order to provide sufficient sterilization of the interior of the vacuum system. The method for sterilization is depicted in FIG. 4 comprising the steps of:

Step S01, closing the main valve 20 in order to separate the vacuum system from the processed chamber connected to the flange 18.

Preferably, if a sterilization valve 24 is implemented, in step S02 the sterilization valve 24 is opened in order to connect the sterilization device 22 to the rest of the vacuum system.

In step S03, a sterilization gas is provided by the sterilization device 22 wherein, due to maintaining operation of the vacuum pump 10, the sterilization gas is drawn from the sterilization device 22 through the pipe 15, through the vacuum pump 10 towards the exhaust 16 of the vacuum system providing sufficient sterilization effect. Therein, of course the steps S01, S02 and S03 can be performed in a different order and could also be performed simultaneously. However, in order to maintain a certain vacuum in the process chamber it might be advantageous to first close the main valve 20 before opening the sterilization valve 24.

In the following it is referred to different examples of the present disclosure wherein in the figures same or similar elements are denoted with the same reference signs.

With respect to the FIGS. 2 and 3 it is only referred to the differences compared to FIG. 1. Referring to FIG. 2 an inlet valve 26 is disposed at the inlet 12 of the vacuum pump 10. Referring to FIG. 3 an outlet valve 28 is disposed at the outlet 14 of the vacuum pump 10. Therein, of course the examples of FIGS. 2 and 3 can be freely combined and the vacuum system according to the present disclosure may comprise an inlet valve 26 as well as an outlet valve 28. The respective method of operation is depicted in FIG. 5 wherein the steps S01 to S03 are the same as in FIG. 4. In addition, in step S04 the inlet valve 26 and/or the outlet valve 28 is at least partially and preferably completely closed thereby reducing or stopping the gas flow through the vacuum system and creating a time of stay or time of retention of the sterilization gas within the vacuum system. After a predetermined length of retention, in step S05 the respective inlet valve 26 and/or outlet valve 28 is reopened and the sterilization gas is conveyed by action of the vacuum pump 10 towards the exhaust 16 of the vacuum system. Thus, by closing the inlet valve 26 and/or the outlet valve 28 a sufficient application time of the sterilization gas to the interior of the vacuum system is ensured. At the same time most of the sterilization gases are highly reactive and degrading with the time. Thus, after a certain time no more sterilization effect is provided by the degraded sterilization gas. Therefore, steps S04 and S05 might be repeated as indicated by arrow 30 in order to improve sterilization effect and provide a sufficient sterilization of the interior of the vacuum system.

A further possible sterilization process could be as followed:
Closing main valve;
Evacuating vacuum system by the vacuum pump;
Stopping operation of the vacuum pump or closing inlet valve;
Opening of sterilization valve and repressurizing the vacuum system between the main valve and the vacuum pump or inlet valve through the sterilization device, thereby providing the sterilization gas to the vacuum system;
Closing the sterilization valve and retention of time; and
Evacuating sterilization gas from the vacuum system using the vacuum pump.

Thus, it is not necessary to continuously provide a sterilization gas by the sterilization device during the retention time. As long as concentration of the sterilization gas within the vacuum system is sufficient for sterilization, no further sterilization gas needs to be provided by the sterilization device and the sterilization valve can be closed. Thereby unnecessary repressurizing of the vacuum system can be avoided.

1. Vacuum system, in particular for food processing, comprising:
a main valve being connectable to a processing chamber,
a vacuum pump having an inlet and an outlet, wherein the inlet is connected to the main valve to connect the vacuum pump with the processing chamber, and
a sterilization device being connected at a point of connection between the main valve and the inlet, wherein the sterilization device is configured to provide a sterilization gas.

2. Vacuum system according to claim 1, wherein a sterilization valve is disposed between the sterilization device and the point of connection.

3. Vacuum system according to claim 1, wherein the sterilization gas is one or more of ozone, fluorine, bromine, iodine, and hydrogen peroxide, all in gaseous form.

4. Vacuum system according to claim 1, wherein sterilization device is an ozone generator configured to generate ozone by a plasma, electrolysis, and electrostatic discharge.

5. Vacuum system according to claim 1, wherein the sterilization gas has a concentration of less than 20%.

6. Vacuum system according to claim 1, wherein an inlet valve is arranged between the sterilization device and the inlet and/or wherein an outlet valve is arranged at the outlet of the vacuum pump, wherein preferably the inlet valve and/or the outlet valve is built as gas flow limiter.

7. Vacuum system according to claim 1, comprising a gas filter arranged at the outlet of the vacuum pump.

8. Vacuum system according to claim 1, wherein the vacuum pump is built as oil-free vacuum pump.

9. Method for sterilization of a vacuum system according to claim 1, the method comprising:
Closing the main valve; and
Starting the sterilization device.

10. Method for sterilization according to claim 9, wherein operation of the vacuum pump is maintained to create a gas flow from the sterilization device towards the vacuum pump.

11. Method for sterilization according to claim 9, wherein the pump performance of the vacuum pump is reduced to reduce gas flow through the vacuum system.

12. Method for sterilization according to claim 9, comprising the step of opening the sterilization valve to connect the sterilization device with the vacuum pump.

13. Method for sterilization according to claim 9, wherein the gas flow is reduced by partially closing the inlet valve and/or the outlet valve.

14. Method for sterilization according to claim 9, comprising:
completely closing the inlet valve and/or the outlet valve,
reopening the inlet valve and/or the outlet valve after a predetermined length of retention, and
repeating periodically.

15. Method for sterilization according to claim 14, wherein the length of retention corresponds to the chemical decay time of ozone.
